(19) United States
(12) Patent Application Publication
(10) Pub. No.: US 2013/0109884 A1
(43) Pub. Date: May 2, 2013

(54) PROCESS FOR PRODUCING ALPHA-KETOCARBOXYLIC ACID

(71) Applicant: Koji Hagiya, Ibaraki-shi (JP)

(72) Inventor: Koji Hagiya, Ibaraki-shi (JP)

(73) Assignee: SUMITOMO CHEMICAL COMPANY, LIMITED, Chuo-ku, Tokyo (JP)

(21) Appl. No.: 13/806,581

(22) PCT Filed: Jun. 21, 2011
(86) PCT No.: PCT/JP2011/064617

§ 371 Date: Dec. 21, 2012

(30) Foreign Application Priority Data
Jun. 25, 2010 (JP) 2010-144589
Aug. 25, 2010 (JP) 2010-187917

(51) Int. Cl. C07C 51/16

(52) U.S. Cl. CPC C07C 51/16
USPC 562/459; 562/531

(57) ABSTRACT

The present invention relates to a process for producing an α-ketocarboxylic acid, comprising a step of oxidizing an α-ketoaldehyde by mixing a base, carbon dioxide, the α-ketoaldehyde and a compound represented by formula (2-1).

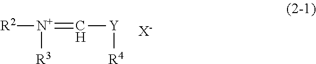

### TECHNICAL FIELD

The present application is filed, claiming the priorities based on the Japanese Patent Application Nos. 2010-144589 (filed on Jun. 25, 2010) and 2010-187917 (filed on Aug. 25, 2010), and a whole of the contents of these applications is incorporated herein by reference.

The present invention relates to a process for producing an α-ketocarboxylic acid.

### BACKGROUND ART

Alpha-ketocarboxylic acids are known as useful compounds for an intermediate in the preparation of pharmaceuticals and agrichemicals since α-ketocarboxylic acids can be converted to α-amino acids by reductive amination.

As a process for producing an α-ketocarboxylic acid, a process in which phenylglyoxal as an α-ketoaldehyde is oxidized with concentrated sulfuric acid and sodium nitrite to obtain benzoylformic acid, is disclosed in J. Mol. Catal. A: Chemical, 2005, 235, pp. 17-25. In addition, a process in which phenylglyoxal is oxidized with dimethyldioxolan to obtain benzoylformic acid, is disclosed in Org. Biomol. Chem., 2005, 3, pp. 2310-2318.

### DISCLOSURE OF INVENTION

### Problem to be Solved by the Invention

An object of the present invention is to provide a new process for producing an α-ketocarboxylic acid.

### Means for Solving the Problem

As a result of the present inventors' intensive studies for solving the above-described problem, the present invention is accomplished.

The present invention provides the followings:
[1] A process for producing an α-ketocarboxylic acid, comprising a step of oxidizing an α-ketoaldehyde by mixing a base, carbon dioxide, the α-ketoaldehyde and a compound represented by formula (2-1):

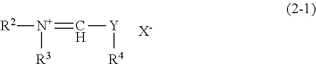

wherein
R² is an alkyl group optionally having a substituent or an aryl group optionally having a substituent;
R³ and R⁴ are independently an alkyl group optionally having a substituent or an aryl group optionally having a substituent, or R³ and R⁴ combine together to form a divalent hydrocarbon group optionally having a substituent or a group of —CH═N— optionally having a substituent;
Y is a group of —S— or —N(R⁵)—, in which R⁵ is an alkyl group optionally having a substituent or an aryl group optionally having a substituent, or R⁵ combines together with R⁴ to form a divalent hydrocarbon group optionally having a substituent; and
X⁻ is an anion.
[2] The process according to the above item [1], wherein the α-ketoaldehyde is a compound represented by formula (1):

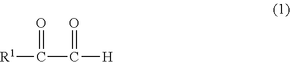

wherein
R¹ is a hydrocarbon group optionally having a substituent or a heteroaryl group optionally having a substituent, and wherein the α-ketocarboxylic acid is a compound represented by formula (3):

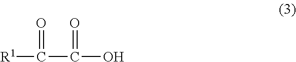

wherein R² means the same as defined above.
[3] The process according to the above item [1] or [2], wherein the compound represented by the formula (2-1) is a compound represented by formula (2-2):

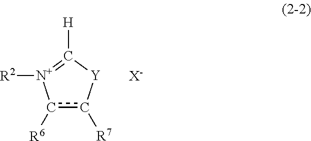

wherein
R² and Y mean the same as defined above;
R⁶ and R⁷ are independently a hydrogen atom, an alkyl group optionally having a substituent or an aryl group optionally having a substituent, or R⁶ and R⁷ are bonded to each other to form a ring together with carbon atoms to which they attach, or R⁷ combines together with R⁵ to form a divalent hydrocarbon group optionally having a substituent;

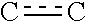

represents a carbon-carbon single bond or a carbon-carbon double bond; and
X⁻ means the same as defined above, or a compound represented by formula (2-3):

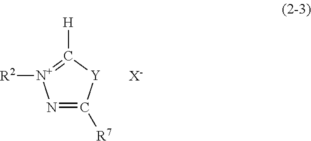

wherein
R² and Y mean the same as defined above;
R⁷ is a hydrogen atom, an alkyl group optionally having a substituent or an aryl group optionally having a substituent, or R⁷ combines together with R⁵ to form a divalent hydrocarbon group optionally having a substituent; and
X⁻ means the same as defined above.
[4] The process according to the above item [3], wherein the compound represented by the formula (2-1) is a compound represented by the formula (2-2).
[5] The process according to the above item [4], wherein in the formula (2-2) Y is a group of —N(R⁵)—, R² and R⁵ are independently a 2,6-disubstituted phenyl group, R⁶ and R⁷ are both a hydrogen atom, and

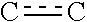

represents a carbon-carbon double bond.
[6] The process according to any one of the above items [1] to [5], wherein the base is at least one base selected from the group consisting of organic bases, alkali metal carbonates and alkaline earth metal carbonates.
[7] A process for producing an α-ketocarboxylic acid, comprising a step of oxidizing an α-ketoaldehyde in the presence of carbon dioxide and a compound obtained by bringing a base into contact with a compound represented by formula (2-1):

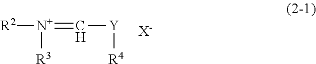

wherein
R² is an alkyl group optionally having a substituent or an aryl group optionally having a substituent;
R³ and R⁴ are independently an alkyl group optionally having a substituent or an aryl group optionally having a substituent, or R³ and R⁴ combine together to form a divalent hydrocarbon group optionally having a substituent or a group of —CH═N— optionally having a substituent;
Y is a group of —S— or —N(R⁵)—, in which R⁵ is an alkyl group optionally having a substituent or an aryl group optionally having a substituent, or R⁵ combines together with R⁴ to form a divalent hydrocarbon group optionally having a substituent; and
X⁻ is an anion.
[8] The process according to the above item [7], wherein the α-ketoaldehyde is a compound represented by formula (1):

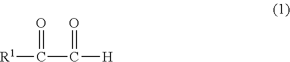

wherein
R¹ is a hydrocarbon group optionally having a substituent or a heteroaryl group optionally having a substituent, and wherein the α-ketocarboxylic acid is a compound represented by formula (3):

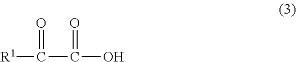

wherein R¹ means the same as defined above.
[9] The process according to the above item [7] or [8], wherein the compound represented by the formula (2-1) is a compound represented by formula (2-2):

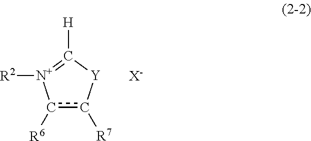

wherein
R² and Y mean the same as defined above;
R⁶ and R⁷ are independently a hydrogen atom, an alkyl group optionally having a substituent or an aryl group optionally having a substituent, or R⁶ and R⁷ are bonded to each other to form a ring together with carbon atoms to which they attach, or R⁷ combines together with R⁵ to form a divalent hydrocarbon group optionally having a substituent;

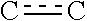

represents a carbon-carbon single bond or a carbon-carbon double bond; and
X⁻ means the same as defined above,
or a compound represented by formula (2-3):

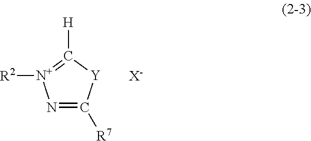

wherein
R² and Y mean the same as defined above;
R⁷ is a hydrogen atom, an alkyl group optionally having a substituent or an aryl group optionally having a substituent, or R⁷ combines together with R⁵ to form a divalent hydrocarbon group optionally having a substituent; and
X⁻ means the same as defined above.
[10] The process according to the above item [9], wherein the compound represented by the formula (2-1) is a compound represented by the formula (2-2).
[11] The process according to the above item [10], wherein in the formula (2-2) Y is a group of —N(R⁵)—, R² and R⁵ are independently a 2,6-disubstituted phenyl group, R⁶ and R⁷ are both a hydrogen atom, and

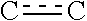

represents a carbon-carbon double bond.
[12] The process according to any one of the above items [7] to [11], wherein the base is at least one base selected from the group consisting of organic bases, alkali metal carbonates and alkaline earth metal carbonates.

According to the present invention, a new process for producing an α-ketocarboxylic acid can be provided.

### MODES FOR CARRYING OUT THE INVENTION

Hereinafter, the present invention will be described in detail.

There is no limit in selection of the α-ketoaldehyde for use in the present invention if it is an aldehyde having a carbonyl group at α-position. As the α-ketoaldehyde, it is preferable to use a compound represented by the formula (1). Hereinafter, the compound represented by the formula (1) is sometimes referred to as a compound (1).

As to R¹ in the formula (1), the hydrocarbon group optionally having a substituent may be an alkyl group optionally having a substituent, an alkenyl group optionally having a substituent and an aryl group optionally having a substituent.

As to R¹, the alkyl group may be linear or branched C₁-C₁₂ alkyl groups such as a methyl group, an ethyl group, a propyl group, an isopropyl group, a butyl group, an isobutyl group, a sec-butyl group, a tert-butyl group, a pentyl group and a decyl group; and C₃-C₁₂ cycloalkyl groups such as a cyclopropyl group, a 2,2-dimethylcyclopropyl group, a cyclopentyl group, a cyclohexyl group and a menthyl group.

Examples of the substituent which the alkyl group may have include a group selected from the following Group 1:

### <Group 1>

a C₁-C₁₀ alkoxy group optionally having a fluorine atom;
a C₇-C₂₀ aralkyloxy group optionally having a C₁-C₁₀ alkoxy group;
a C₇-C₂₀ aralkyloxy group having a C₆-C₁₀ aryloxy group;
a C₆-C₁₀ aryloxy group optionally having a C₁-C₁₀ alkoxy group;
a C₆-C₁₀ aryloxy group having a C₆-C₁₀ aryloxy group;
a C₂-C₁₀ acyl group optionally having a C₁-C₁₀ alkoxy group;
a C₁-C₁₀ alkylthio group;
a C₂-C₁₀ alkoxycarbonyl group; and
a halogen atom.

In the Group 1, examples of the C₁-C₁₀ alkoxy group optionally having a fluorine atom include a methoxy group, an ethoxy group, a propoxy group, an isopropoxy group, a butoxy group, an isobutoxy group, a sec-butoxy group, a tert-butoxy group and a trifluoromethyloxy group.

Examples of the C₇-C₂₀ aralkyloxy group optionally having a C₁-C₁₀ alkoxy group include a benzyloxy group, a 4-methylbenzyloxy group and a 4-methoxybenzyloxy group.

Examples of the C₇-C₂₀ aralkyloxy group having a C₆-C₁₀ aryloxy group include a 3-phenoxybenzyloxy group.

Examples of the C₆-C₁₀ aryloxy group optionally having a C₁-C₁₀ alkoxy group include a phenoxy group, a 2-methylphenoxy group, a 4-methylphenoxy group and a 4-methoxyphenoxy group.

Examples of the C₆-C₁₀ aryloxy group having a C₆-C₁₀ aryloxy group include a 3-phenoxyphenoxy group.

Examples of the C₂-C₁₀ acyl group optionally having a C₁-C₁₀ alkoxy group include an acetyl group, a propionyl group, a benzylcarbonyl group, a 4-methylbenzylcarbonyl group, a 4-methoxybenzylcarbonyl group, a benzoyl group, 2-methylbenzoyl group, a 4-methylbenzoyl group and 4-methoxybenzoyl group.

Examples of the C₁-C₁₀ alkylthio group include a methylthio group, an ethylthio group and an isopropylthio group.

Examples of the C₂-C₁₀ alkoxycarbonyl group include a methoxycarbonyl group and an ethoxycarbonyl group.

Examples of the halogen atom include a fluorine atom, a chlorine atom and a bromine atom.

Examples of the alkyl group having a group selected from Group 1 include a chloromethyl group, a fluoromethyl group, a trifluoromethyl group, a methoxymethyl group, an ethoxymethyl group, a methoxyethyl group, a methoxycarbonylmethyl group, a 1-ethoxycarbonyl-2,2-dimethyl-3-cyclopropyl group and a 2-methylthioethyl.

As to R¹, the alkenyl group may be linear or branched C₂-C₁₂ alkenyl groups such as a vinyl group, a 1-propenyl group, 1-butenyl group, 2-methyl-1-propenyl group; and C₃-C₁₂ cycloalkenyl group such as 1-cyclohexenyl group.

Examples of the substituent which the alkenyl group may have include a group selected from the above Group 1.

Examples of the alkenyl group having a group selected from Group 1 include a 2-chlorovinyl group and a 2-trifluoromethylvinyl group.

As to R¹, the aryl group may be C₆-C₂₀ aryl groups such as a phenyl group, a 2-methylphenyl group, a 4-methylphenyl group, a 1-naphthyl group, a 2-naphthyl group and a styryl group.

Examples of the substituent which the aryl group may have include a group selected from the following Group 2:

### <Group 2>

a C₁-C₁₀ alkoxy group optionally having a fluorine atom or a C₁-C₁₀ alkoxy group;
a C₆-C₁₀ aryloxy group optionally having a C₁-C₁₀ alkoxy group;
a C₆-C₁₀ aryloxy group having a C₆-C₁₀ aryloxy group;
a C₂-C₁₀ acyl group optionally having a C₁-C₁₀ alkoxy group;
a C₁-C₆ alkylenedioxy group;
a nitro group; and
a halogen atom.

In the Group 2, examples of the C₁-C₁₀ alkoxy group optionally having a fluorine atom or a C₁-C₁₀ alkoxy group include a methoxy group, an ethoxy group, a propoxy group, an isopropoxy group, a butoxy group, an isobutoxy group, a sec-butoxy group, a tert-butoxy group, a pentyloxy group, a cyclopentyloxy group, fluoromethoxy group, a trifluoromethoxy group, a methoxymethoxy group, an ethoxymethoxy group and a methoxyethoxy group.

Examples of the C₆-C₁₀ aryloxy group optionally having a C₁-C₁₀ alkoxy group include a phenoxy group, a 2-methylphenoxy group, a 4-methylphenoxy group and a 4-methoxyphenoxy group.

Examples of the C₆-C₁₀ aryloxy group having a C₆-C₁₀ aryloxy group include a 3-phenoxyphenoxy group.

Examples of the C₂-C₁₀ acyl group optionally having a C₁-C₁₀ alkoxy group include an acetyl group, a propionyl group, a benzylcarbonyl group, a 4-methylbenzylcarbonyl group and a 4-methoxybenzylcarbonyl group.

Examples of the C₁-C₆ alkylenedioxy group include a methylenedioxy group and an ethylendioxy group.

Examples of the halogen atom include a fluorine atom and a chlorine atom.

Examples of the aryl group having a group selected from Group 2 include a 4-chlorophenyl group, a 4-methoxyphenyl group and a 3-phenoxyphenyl group.

As to R¹, the heteroaryl group may be C₄-C₁₀ heteroaryl group which has at least one hetero atom such as a nitrogen atom, an oxygen atom and a sulfur atom. The specific examples of the heteroaryl group include a 2-pyridyl group, a 3-pyridyl group, a 4-pyridyl group, a 2-furyl group, a 3-furyl group, a 5-methyl-2-furyl group and 2-chloro-3-pyridinyl group.

Examples of the compound (1) include phenylglyoxal, 4-chlorophenylglyoxal, 2-methylphenylglyoxal, 4-fluoro phenylglyoxal, 2-methoxyphenylglyoxal, 2,4-dichlorophenylglyoxal, 2-nitrophenylglyoxal, 2-naphthylglyoxal, 2-pyridineglyoxylaldehyde, methylglyoxal, ethylglyoxal, n-propylglyoxal, isopropylglyoxal, cyclohexylglyoxal, 4-(methylthio)-2-oxo-1-butanal, vinylglyoxal and styrylglyoxal.

As the compound (1), a commercially available product may be used. And also the compound (1) can be synthesized according to a known method, such as a method in which ketoalcohol is oxygen oxidized in the presence of a metal catalyst. Such method can be found in JP 2000-336055 A, for example.

The step of oxidizing an α-ketoaldehyde (hereinafter sometimes referred to as “the present reaction”) may be carried out by mixing a base, carbon dioxide, an α-ketoaldehyde and a compound represented by the formula (2-1). The present reaction may be carried out in the presence of carbon dioxide and a compound obtained by bringing a base into contact with a compound represented by the formula (2-1).

Hereinafter, the compound represented by the formula (2-1) (hereinafter sometimes referred to as a compound (2-1)) for use in the present invention is described.

As to R³ and R⁴ in the formula (2-1), the alkyl group may be linear or branched C₁-C₁₂ alkyl groups such as a methyl group, an ethyl group, a propyl group, an isopropyl group, a butyl group, an isobutyl group, a sec-butyl group, a tert-butyl group, a pentyl group and a decyl group; and C₃-C₁₂ cycloalkyl groups such as a cyclopropyl group, a 2,2-dimethylcyclopropyl group, a cyclopentyl group, a cyclohexyl group and a menthyl group.

Examples of the substituent which the alkyl group may have include a group selected from the following Group 3:

### <Group 3>

a C₆-C₁₀ aryl group optionally having a C₁-C₁₀ alkoxy group;
a C₁-C₁₀ alkoxy group optionally having a fluorine atom;
a benzyloxy group optionally having at least one group selected from the group consisting of C₁-C₁₀ alkoxy group, C₁-C₁₀ alkyl group and C₆-C₁₀ aryloxy group;
a C₆-C₁₀ aryloxy group optionally having a C₁-C₁₀ alkoxy group;
a C₆-C₁₀ aryloxy group optionally having a C₆-C₁₀ aryloxy group;
a C₂-C₁₀ acyl group optionally having a C₁-C₁₀ alkoxy group;
a carboxy group; and
a fluorine atom.

In the Group 3, examples of the C₆-C₁₀ aryl group optionally having a C₁-C₁₀ alkoxy group include a phenyl group, a naphthyl group, 4-methylphenyl group and 4-methoxyphenyl group.

Examples of the C₁-C₁₀ alkoxy group optionally having a fluorine atom include a methoxy group, an ethoxy group, a propoxy group, an isopropoxy group, a butoxy group, an isobutoxy group, a sec-butoxy group, a tert-butoxy group and a trifluoromethyloxy group.

Examples of the benzyloxy group optionally having at least one group selected from the group consisting of C₁-C₁₀ alkoxy group, C₁-C₁₀ alkyl group and C₆-C₁₀ aryloxy group include a benzyloxy group, a 4-methylbenzyloxy group, a 4-methoxybenzyloxy group and a 3-phenoxybenzyloxy group.

Examples of the C₆-C₁₀ aryloxy group optionally having a C₁-C₁₀ alkoxy group include a phenoxy group, a 2-methylphenoxy group, a 4-methylphenoxy group and a 4-methoxyphenoxy group.

Examples of the C₆-C₁₀ aryloxy group having a C₆-C₁₀ aryloxy group include a 3-phenoxyphenoxy group.

Examples of the C₂-C₁₀ acyl group optionally having a C₁-C₁₀ alkoxy group include an acetyl group, a propionyl group, a benzylcarbonyl group, a 4-methylbenzylcarbonyl group, a 4-methoxybenzylcarbonyl group, a benzoyl group, 2-methylbenzoyl group, a 4-methylbenzoyl group and 4-methoxybenzoyl group.

Examples of the alkyl group having a group selected from Group 3 include a fluoromethyl group, a trifluoromethyl group, a methoxymethyl group, an ethoxymethyl group, a methoxyethyl group, a benzyl group, a 4-fluorobenzyl group, a 4-methylbenzyl group, a phenoxymethyl group, a 2-oxopropyl group, a 2-oxobutyl group, a phenacyl group and a 2-carboxyethyl group.

As to R³ and R⁴ in the formula (2-1), the aryl group may be C₆-C₁₀ aryl groups such as a phenyl group, a 2-methylphenyl group, a 4-methylphenyl group and a naphthyl group.

Examples of the substituent which the aryl group may have include a group selected from the above Group 2.

Examples of the aryl group having a group selected from Group 2 include a 4-chlorophenyl group and 4-methoxyphenyl group.

In the formula (2-1), R³ and R⁴ may combine together to form a divalent hydrocarbon group optionally having a substituent or a group of —CH═N— optionally having a substituent. Examples of the divalent hydrocarbon group include an ethylene group, a vinylidene group, a cyclopentane-1,2-diyl group, a cyclohexane-1,2-diyl group and an o-phenylene group. Examples of the substituent which the divalent hydrocarbon group may have include an alkyl group optionally having a substituent and an aryl group optionally having a substituent. Examples of the substituent which the group of —CH═N— may have include an alkyl group optionally having a substituent and an aryl group optionally having a substituent. Examples of the alkyl group include linear or branched C₁-C₁₂ alkyl groups such as a methyl group, an ethyl group, a propyl group, an isopropyl group, a butyl group, an isobutyl group, a sec-butyl group, a tert-butyl group, a pentyl group and a decyl group; and C₃-C₁₂ cycloalkyl groups such as a cyclopropyl group, a 2,2-dimethylcyclopropyl group, a cyclopentyl group, a cyclohexyl group and a menthyl group. Examples of the aryl group include C₆-C₁₀ aryl groups such as a phenyl group, a 2-methylphenyl group, a 4-methylphenyl group and a naphthyl group.

As to R² and R⁵ in the formula (2-1), the alkyl group may be linear or branched C₁-C₁₂ alkyl groups such as a methyl group, an ethyl group, a propyl group, an isopropyl group, a butyl group, an isobutyl group, a sec-butyl group, a tert-butyl group, a pentyl group, a tert-pentyl group and a decyl group; and C₃-C₁₂ cycloalkyl groups such as a cyclopropyl group, a 2,2-dimethylcyclopropyl group, a cyclopentyl group, a cyclohexyl group, a menthyl group and an adamantyl group.

Examples of the substituent which the alkyl group may have include a group selected from the following Group 4:

### <Group 4>

a C₆-C₁₀ aryl group optionally having a C₁-C₁₀ alkoxy group;
a C₁-C₁₀ alkoxy group optionally having a fluorine atom;
a C₇-C₂₀ aralkyloxy group optionally having a C₁-C₁₀ alkoxy group;
a C₇-C₂₀ aralkyloxy group having a C₆-C₁₀ aryloxy group;
a C₆-C₁₀ aryloxy group optionally having a C₁-C₁₀ alkoxy group;
a C₆-C₁₀ aryloxy group having a C₆-C₁₀ aryloxy group; and
a C₂-C₁₀ acyl group optionally having a C₁-C₁₀ alkoxy group.

In the Group 4, examples of the C₆-C₁₀ aryl group optionally having a C₁-C₁₀ alkoxy group include a phenyl group, a naphthyl group, 4-methylphenyl group and 4-methoxyphenyl group.

Examples of the C₁-C₁₀ alkoxy group optionally having a fluorine atom include a methoxy group, an ethoxy group, a propoxy group, an isopropoxy group, a butoxy group, an isobutoxy group, a sec-butoxy group, a tert-butoxy group and a trifluoromethyloxy group.

Examples of the C₇-C₂₀ aralkyloxy group optionally having a C₁-C₁₀ alkoxy group include a benzyloxy group, a 4-methylbenzyloxy group and a 4-methoxybenzyloxy group.

Examples of the C₇-C₂₀ aralkyloxy group having a C₆-C₁₀ aryloxy group include a 3-phenoxybenzyloxy group.

Examples of the C₆-C₁₀ aryloxy group optionally having a C₁-C₁₀ alkoxy group include a phenoxy group, a 2-methylphenoxy group, a 4-methylphenoxy group and a 4-methoxyphenoxy group.

Examples of the C₆-C₁₀ aryloxy group having a C₆-C₁₀ aryloxy group include a 3-phenoxyphenoxy group.

Examples of the C₂-C₁₀ acyl group optionally having a C₁-C₁₀ alkoxy group include an acetyl group, a propionyl group, a benzylcarbonyl group, a 4-methylbenzylcarbonyl group, a 4-methoxybenzylcarbonyl group, a benzoyl group, 2-methylbenzoyl group, a 4-methylbenzoyl group and 4-methoxybenzoyl group.

Examples of the alkyl group having a group selected from Group 4 include a methoxymethyl group, an ethoxymethyl group, a methoxyethyl group, a benzyl group, 4-fluorobenzyl group, a 4-methylbenzyl group, a phenoxymethyl group, a 2-oxopropyl group, 2-oxobutyl group and a phenacyl group.

As to R² and R⁵ in the formula (2-1), the aryl group may be C₆-C₂₀ aryl groups such as a phenyl group, a naphthyl group, a 2-methylphenyl group, a 4-methylphenyl group, a 2,6-dimethylphenyl group, a 2,4,6-trimethylphenyl group and a 2,6-diisopropylphenyl group.

Examples of the substituent which the aryl group may have include a group selected from the following Group 5:

### <Group 5>

a C₁-C₁₀ alkoxy group optionally having a fluorine atom or a C₁-C₁₀ alkoxy group; and
a halogen atom.

In the Group 5, examples of the C₁-C₁₀ alkoxy group optionally having a fluorine atom or a C₁-C₁₀ alkoxy group include a methoxy group, an ethoxy group, a propoxy group, an isopropoxy group, a butoxy group, an isobutoxy group, a sec-butoxy group, a tert-butoxy group, a pentyloxy group, a cyclopentyloxy group, fluoromethoxy group, a trifluoromethoxy group, a methoxymethoxy group, an ethoxymethoxy group and a methoxyethoxy group.

Examples of the halogen atom include a fluorine atom and a chlorine atom.

Examples of the aryl group having a group selected from Group 5 include a 4-chlorophenyl group, a 4-methoxyphenyl group and a 2,6-dichlorophenyl group.

In the formula (2-1), R⁴ and R⁵ may combine together to form a divalent hydrocarbon group optionally having a substituent. Examples of the divalent hydrocarbon group include polymethylene groups such as an ethylene group, a trimethylene group and a tetramethylene group, a vinylidene group, a cyclopentane-1,2-diyl group, a cyclohexane-1,2-diyl group and an o-phenylene group. Examples of the substituent which the divalent hydrocarbon group may have include an alkyl group optionally having a substituent and an aryl group optionally having a substituent. Examples of the alkyl group include linear or branched C₁-C₁₂ alkyl groups such as a methyl group, an ethyl group, a propyl group, an isopropyl group, a butyl group, an isobutyl group, a sec-butyl group, a tert-butyl group, a pentyl group and a decyl group; and C₃-C₁₂ cycloalkyl groups such as a cyclopropyl group, a 2,2-dimethylcyclopropyl group, a cyclopentyl group, a cyclohexyl group and a menthyl group. Examples of the aryl group include C₆-C₁₀ aryl groups such as a phenyl group, a 2-methylphenyl group, a 4-methylphenyl group and a naphthyl group.

In the formula (2-1), the anion represented by X⁻ may be halide ion such as a chloride ion, a bromide ion and an iodide ion; alkanesulfonate ions optionally having a fluorine atom, such as a methanesulfonate and a trifluoromethanesulfonate; acetate ions optionally having a halogen atom, such as a trifluoroacetate and a trichloroacetate; a nitrate ion; a perchlorate ion; tetrahaloborate ions such as a tetrafluoroborate and a tetrachloroborate; hexahalophosphate ions such as a hexafluorophosphate; hexahaloantimonate ions such as a hexafluoroantimonate and a hexachloroantimonate; pentahalostannate ions such as a pentafluorostannate and a pentachlorostannate; and tetraarylborate ion optionally having a substituent, such as a tetraphenylborate, a tetrakis(pentafluorophenyl)borate, a tetrakis[3,5-bis(trifluoromethyl)phenyl]borate.

The compound (2-1) is preferably a compound represented by the formula (2-2) (hereinafter sometimes referred to as a compound (2-2)) or a compound represented by the formula (2-3) (hereinafter sometimes referred to as a compound (2-3)), more preferably a compound (2-2).

The present reaction is carried out preferably by mixing the compound (2-2) or (2-3), a base, carbon dioxide and an α-ketoaldehyde. More preferably, the present reaction is carried out by mixing the compound (2-2), a base, carbon dioxide and an α-ketoaldehyde. In addition, the present reaction is carried out preferably in the presence of carbon dioxide and a compound obtained by bringing a base into contact with the compound (2-2) or (2-3), more preferably in the presence of carbon dioxide and a compound obtained by bringing a base into contact with the compound (2-2).

Hereinafter, the compounds (2-2) and (2-3) will be described.

In the formulae (2-2) and (2-3), R² and Y mean the same as defined in the formula (2-1). When Y is a group of —N(R⁵)— in the formulae (2-2) and (2-3), R⁵ means the same as defined in the formula (2-1). X⁻ means the same as defined in the formula (2-1).

In the formulae (2-2) and (2-3), Y is preferably a group of —N(R⁵)—.

In the formula (2-2), it is preferred that at least one of R² and R⁵ is a bulky group. More preferably, R² and R⁵ are both a bulky group. R² and R⁵ may be the same group or may be a different group from each other.

As to R² and R⁵, examples of the bulky group include C₄-C₁₂ tertiary alkyl groups such as a tert-butyl group and a tert-pentyl group; C₃-C₁₀ cycloalkyl groups such as a cyclopropyl group, a 2,2-dimethylcyclopropyl group, a cyclopentyl group, a cyclohexyl group, a menthyl group and an adamantyl group; phenyl groups having substituents at least on 2-position and 6-position, i.e. 2,6-disubstituted phenyl group, such as a 2,6-dimethylphenyl group, a 2,6-dichlorophenyl group, 2,4,6-trimethylphenyl group and 2,6-diisopropylphenyl group; and naphthyl groups having a C₁-C₁₀ alkyl group at 2-position, such as a 2-methylnaphthyl group. As the substituent included in 2,6-disubstituted phenyl group, a C₁-C₁₂ alkyl group and halogen atoms are exemplified.

The bulky group is preferably a tert-butyl group, a tert-pentyl group, a cyclohexyl group, an adamantyl group or a 2,6-disubstituted phenyl group, more preferably a 2,6-disubstituted phenyl group, still more preferably a 2,6-diisopropylphenyl group.

As to R⁶ in the formula (2-2) and R⁷ in the formulae (2-2) and (2-3), the alkyl group may be linear or branched C₁-C₁₀ alkyl groups such as a methyl group, an ethyl group, a propyl group, an isopropyl group, a butyl group, an isobutyl group, a sec-butyl group, a tert-butyl group, a pentyl group and a decyl group; and C₃-C₁₀ cycloalkyl groups such as a cyclopropyl group, a 2,2-dimethylcyclopropyl group, a cyclopentyl group, a cyclohexyl group and a menthyl group.

Examples of the substituent which the alkyl group may have include a group selected from the above Group 3.

Examples of the alkyl group having a group selected from Group 3 include a fluoromethyl group, a trifluoromethyl group, a methoxymethyl group, an ethoxymethyl group, a methoxyethyl group, a benzyl group, a 4-fluorobenzyl group, a 4-methylbenzyl group, a phenoxymethyl group, a 2-oxopropyl group, a 2-oxobutyl group, a phenacyl group and a 2-carboxyethyl group.

As to R⁶ in the formula (2-2) and R⁷ in the formulae (2-2) and (2-3), the aryl group may be C₆-C₁₀ aryl groups such as a phenyl group, a 2-methylphenyl group, a 4-methylphenyl group and a naphthyl group.

Examples of the substituent which the aryl group may have include a group selected from the above Group 2.

Examples of the aryl group having a group selected from Group 2 include a 4-chlorophenyl group and a 4-methoxyphenyl group.

In the formula (2-2), R⁶ and R⁷ may be bonded to each other to form a ring together with carbon atoms to which they attach. Examples of such a ring include a cyclopentane ring, a cyclohexane ring and a benzene ring.

In the formula (2-2), preferably, R⁶ and R⁷ are independently a hydrogen atom or an alkyl group optionally having a substituent, more preferably, R⁶ and R⁷ are both a hydrogen atom.

In the formulae (2-2) and (2-3), R⁵ and R⁷ may combine together to form a divalent hydrocarbon group optionally having a substituent. Examples of the divalent hydrocarbon group include polymethylene groups such as an ethylene group, a trimethylene group and a tetramethylene group, a vinylidene group, a cyclopentane-1,2-diyl group, a cyclohexane-1,2-diyl group and an o-phenylene group. Examples of the substituent which the divalent hydrocarbon group may have include an alkyl group optionally having a substituent and an aryl group optionally having a substituent. Examples of the alkyl group include linear or branched C₁-C₁₂ alkyl groups such as a methyl group, an ethyl group, a propyl group, an isopropyl group, a butyl group, an isobutyl group, a sec-butyl group, a tert-butyl group, a pentyl group and a decyl group; and C₃-C₁₂ cycloalkyl groups such as a cyclopropyl group, a 2,2-dimethylcyclopropyl group, a cyclopentyl group, a cyclohexyl group and a menthyl group. Examples of the aryl group include C₆-C₁₀ aryl groups such as a phenyl group, a 2-methylphenyl group, a 4-methylphenyl group and a naphthyl group.

In the formula (2-2),

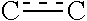

preferably represents a carbon-carbon double bond.

The specific examples of the compound (2-2) include 1,3-di-tert-butylimidazolium chloride, 1,3-di-tert-butylimidazolinium chloride, 1,3-dicyclohexylimidazolium chloride, 1,3-dicyclohexylimidazolinium chloride, 1,3-diadamantylimidazolium chloride, 1,3-diadamantylimidazolinium chloride, 1,3-diphenylimidazolium chloride, 1,3-diphenylimidazolinium chloride, 1,3-bis[(2,6-diisopropyl)phenyl]imidazolium chloride, 1,3-bis[(2,6-diisopropyl)phenyl]imidazolinium chloride, 1,3-bis[(2,4,6-trimethyl)phenyl]imidazolium chloride, 1,3-bis[(2,4,6-trimethyl)phenyl]imidazolinium chloride, 4,5-dimethyl-1,3-bis[(2,4,6-trimethyl)phenyl]imidazolium chloride, 4,5-dimethyl-1,3-bis[(2,4,6-trimethyl)phenyl]imidazolinium chloride, 4,5-dimethyl-1,3-bis[(2,6-diisopropyl)phenyl]imidazolium chloride, 4,5-dimethyl-1,3-bis[(2,6-diisopropyl)phenyl]imidazolinium chloride, 4,5-dichloro-1,3-bis[(2,6-diisopropyl)phenyl]imidazolium chloride, 4,5-dichloro-1,3-bis[(2,6-diisopropyl)phenyl]imidazolinium chloride, 4,5-diphenyl-1,3-bis[(2,4,6-trimethyl)phenyl]imidazolium chloride, 4,5-diphenyl-1,3-bis[(2,4,6-trimethyl)phenyl]imidazolinium chloride, 4,5-difluoro-1,3-bis[(2,6-diisopropyl)phenyl]imidazolium chloride, 4,5-difluoro-1,3-bis[(2,6-diisopropyl)phenyl]imidazolinium chloride, 4-methyl-1,3-bis[(2,4,6-trimethyl)phenyl]imidazolium chloride, 4-methyl-1,3-bis[(2,4,6-trimethyl)phenyl]imidazolinium chloride, 1,3-bis[(2,6-dichlor)phenyl]imidazolium chloride, 1,3-bis[(2,6-dichlor)phenyl]imidazolinium chloride, 1-tert-butyl-3-phenylimidazolium chloride, 1-tert-butyl-3-phenylimidazolinium chloride, 1-cyclohexyl-3-[(2,6-diisopropyl)phenyl]imidazolium chloride, 1-cyclohexyl-3-[(2,6-diisopropyl)phenyl]imidazolinium chloride, 1-phenyl-3-[(2,4,6-trimethyl)phenyl]imidazolium chloride, 1-phenyl-3-[(2,4,6-trimethyl)phenyl]imidazolinium chloride, 1-tert-butyl-3-[(2,6-diisopropyl)phenyl]imidazolium chloride, 1-tert-butyl-3-[(2,6-diisopropyl)phenyl]imidazolinium chloride, 1-tert-butyl-3-[(2,4,6-trimethyl)phenyl]imidazolium chloride, 1-tert-butyl-3-[(2,4,6-trimethyl)phenyl]imidazolinium chloride, 3-(2,6-diisopropyl)phenyl-4,5-dimethylthiazolium chloride, 3-phenyl-4,5-dimethylthiazolium chloride, 3-benzylthiazolium chloride and 3-(2,4,6-trimethyl)phenyl-4,5-dimethylthiazolium chloride.

The specific examples of the compound (2-3) include 1,4-dimethyl-1H-1,2,4-triazol-4-ium chloride, 1,3,4-triphenyl-1H-1,2,4-triazol-4-ium chloride and 6,7-dihydro-2-pentafluorophenyl-5H-pyrrolo[2,1-c]-1,2,4-triazolium chloride.

In addition, as the compounds (2-2) and (2-3), the compounds in which “chloride” of the above compounds is substituted by “iodide”, “bromide”, “methanesulfonate”, “trifluoromethanesulfonate”, “nitrate”, “perchlorate”, “tetrafluoroborate”, “tetrachloroborate”, “hexafluorophosphate”, “hexafluoroantimonate”, “hexachloroantimonate”, “pentafluorostannate”, “pentachlorostannate”, “tetraphenylborate”, “tetrakis(pentafluorophenyl)borate” or “tetrakis[3,5-bis(trifluoromethyl)phenyl]borate” are exemplified.

As the compound (2-1), a commercially available product may be used. And also the compound (2-1) can be synthesized according to the methods described in J. Organometallic. Chem. Soc., 606, 49 (2000), J. Organometallic. Chem. Soc., 73, 2784 (2008), or the like.

The amount of the compound (2-1) to be used is preferably from 0.001 to 0.5 mol, more preferably from 0.01 to 0.3 mol per mole of α-ketoaldehyde.

The base for use in the present reaction is preferably at least one base selected from the group consisting of organic bases, alkali metal carbonates and alkaline earth metal carbonates.

Examples of the organic bases include tertiary amine such as triethylamine, trioctylamine, diisopropylethylamine and 4-dimethylaminopyridine; nitrogen-containing alicyclic compounds such as 1,8-diazabicyclo[5.4.0]-7-undecene and 1,5,7-triazabicyclo[4,4,0]-5-decene; nitrogen-containing aromatic compounds such as pyridine and imidazole; and alkali metal alkoxide such as sodium methoxide and sodium ethoxide.

Examples of the alkali metal carbonates include sodium carbonate, sodium bicarbonate, potassium carbonate, potassium bicarbonate, lithium carbonate and lithium bicarbonate.

Examples of the alkaline earth metal carbonates include magnesium carbonate and calcium carbonate.

As the base for use in the present reaction, the organic bases are more preferable.

The amount of the base to be used is usually from 0.001 to 3 mol, preferably from 0.001 to 0.5 mol, more preferably from 0.01 to 0.3 mol per mole of α-ketoaldehyde.

The carbon dioxide for use in the present reaction may be in either form of gaseous carbon dioxide, a solid carbon dioxide (i.e. dry ice) or supercritical carbon dioxide. The gaseous carbon dioxide may be diluted with an inert gas such as nitrogen.

The amount of the carbon dioxide to be used is preferably one mole or more per mole of α-ketoaldehyde. Although the upper limit of the amount is not limited, it is usually 100 mol or less from the viewpoint of productivity.

The present reaction may be carried out further in the presence of a solvent.

There is no limit in selection of the organic solvent if it does not hinder the present reaction. Examples of the solvent include ether solvents such as tetrahydrofuran, methyl-tert-butyl ether, cyclopentyl methyl ether and diisopropyl ether; ester solvents such as ethyl acetate and butyl acetate; aromatic solvents such as toluene and chlorobenzene; nitrile solvents such as acetonitrile and propionitrile; and a mixture thereof.

The amount of the solvent to be used is usually 100 parts by weight or less per part by weight of α-ketoaldehyde, while this amount is not limited.

In the present reaction, the order of blending the reactants is not limited. In the preferred embodiment, for example, α-ketoaldehyde, the compound (2-1) and carbon dioxide, optionally a solvent are mixed, and then, a base is added to the resultant mixture. This mixing is preferably carried out under an atmosphere of an inert gas such as nitrogen.

The present reaction may be carried out under reduced pressure or normal pressure or increased pressure. Preferably, the present reaction is carried out under normal pressure or increased pressure.

A temperature for the present reaction may vary depending on the kind and amount of the compound (2-1) and the base to be used, and is preferably from −20 to 150° C., more preferably from 0 to 100° C. When the reaction temperature is −20° C. or higher, the oxidation reaction rate tends to become higher. When the reaction temperature is 150° C. or lower, the oxidation reaction can be carried out with a higher selectivity.

Progress of the present reaction can be confirmed by analytical means such as gas chromatography, high-performance liquid chromatography, thin-layer chromatography, nucleic magnetic resonance spectrum analysis, or infrared absorption spectrum analysis.

After completion of the reaction, α-ketocarboxylic acid may be brought out by a procedure in which the resultant reaction mixture is optionally neutralized with mineral acid such as sulfuric acid, hydrochloric acid or the like and then concentrated and cooled. Alternatively, α-ketocarboxylic acid may be brought out by a procedure in which an aqueous alkali solution such as aqueous sodium hydroxide is added to the resultant reaction mixture to prepare an aqueous solution of an alkali salt of α-ketocarboxylic acid, and then the resulting aqueous alkaline salt solution is washed with a solvent immiscible to water and is then neutralized, and extracted and/or crystallized.

The solvent immiscible to water may be ester solvents such as ethyl acetate, and ether solvents such as methyl tert-butyl ether. The amount of the immiscible solvent to be used is not limited.

The obtained α-ketocarboxylic acid may be purified by distillation, column chromatography, crystallization or the like.

According to the process of the present invention, for example, the following α-ketocarboxylic acids can be produced: benzoylformic acid, 4-chloro-benzoylformic acid, 2-methylbenzoylformic acid, 4-fluoro-benzoylformic acid, 4-methoxy-benzoylformic acid, 2-nitro-benzoylformic acid, 2,4-dichloro-benzoylformic acid, 2-naphthoylformic acid, α-oxo-2-pyridineacetic acid, pyruvic acid, 2-oxobutanoic acid, 2-oxopentanoic acid, 3-methyl-2-oxobutanoic acid, α-oxo-cyclohexaneacetic acid, 4-(methylthio)-2-oxo-butanoic acid, 2-oxo-3-butenoic acid and 2-oxo-4-phenyl-3-butenoic acid.

### EXAMPLES

Hereinafter, the present invention will be described in more detail by way of Examples.

### Example 1

A 50 ml schrenck tube equipped with a magnetic rotor was charged with phenylglyoxal monohydrate (260 mg), 1,3-bis[(2,6-diisopropyl)phenyl]imidazolium chloride (50 mg) and tetrahydrofuran (5 g) under a nitrogen atmosphere, and the resulting mixture was stirred while maintaining the temperature of the mixture in a water bath at 25° C. Dry ice (1.0 g) was added to the mixture, and 1,8-diazabicyclo[5,4,0]-7-undecene (23 mg) was further added thereto so as to initiate the reaction, and the mixture was stirred for 2 hours at a room temperature. At 30 minutes and 1 hour following the start of reaction, dry ice (1 g) was added to the reaction mixture, respectively. After completion of the reaction, the solvent was distilled off from the reaction mixture to obtain a yellow solid containing benzoylformic acid.

### Determination of Yield

Methanol (5 g) was added to the obtained yellow solid, and a 10% hexane solution of trimethylsilyldiazomethane was further added thereto, to obtain methyl benzoylformate. A methanol solution containing the obtained methyl benzoylformate was analyzed with a gas chromatography internal standard method to determine the yield of methyl benzoylformate from phenylglyoxal. As a result, the yield was 68%. In other words, benzoylformic acid was obtained from phenylglyoxal at a yield of 68% or more.

### Example 2

A 100 ml stainless-steel pressure reaction tube equipped with a magnetic rotor was charged with phenylglyoxal monohydrate (260 mg), 1,3-bis[(2,6-diisopropyl)phenyl]imidazolium chloride (50 mg) and tetrahydrofuran (5 g) under a nitrogen atmosphere, and the resulting mixture was cooled in a dry ice bath at −70° C. After dry ice (2 g) and potassium carbonate (520 mg) were added to the cooled mixture, the pressure reaction tube was sealed. The reaction was carried out by stirring the resulting mixture for 2 hours at 60° C. After completion of the reaction, the solvent was distilled off from the reaction mixture to obtain a yellow solid containing benzoylformic acid.

### Determination of Yield

Methanol (5 g) was added to the obtained yellow solid, and a 10% hexane solution of trimethylsilyldiazomethane was further added thereto, to obtain methyl benzoylformate. A methanol solution containing the obtained methyl benzoylformate was analyzed with a gas chromatography internal standard method to determine the yield of methyl benzoylformate from phenylglyoxal. As a result, the yield was 35%. In other words, benzoylformic acid was obtained from phenylglyoxal at a yield of 35% or more.

### Example 3

A 100 ml stainless-steel pressure reaction tube equipped with a magnetic rotor was charged with phenylglyoxal monohydrate (200 mg), 1,3-bis[(2,6-diisopropyl)phenyl]imidazolium chloride (30 mg) and tetrahydrofuran (5 g) under a nitrogen atmosphere, and the resulting mixture was cooled in a dry ice bath at −70° C. After dry ice (2 g) and sodium bicarbonate (50 mg) were added to the cooled mixture, the pressure reaction tube was sealed. The reaction was carried out by stirring the resulting mixture for 6 hours at 60° C. After completion of the reaction, the solvent was distilled off from the reaction mixture to obtain a yellow solid containing benzoylformic acid.

### Determination of Yield

Methanol (5 g) was added to the obtained yellow solid, and a 10% hexane solution of trimethylsilyldiazomethane was further added thereto, to obtain methyl benzoylformate. A methanol solution containing the obtained methyl benzoylformate was analyzed with a gas chromatography internal standard method to determine the yield of methyl benzoylformate from phenylglyoxal. As a result, the yield was 50%. In other words, benzoylformic acid was obtained from phenylglyoxal at a yield of 50% or more.

### Example 4

A 50 ml schrenck tube equipped with a magnetic rotor was charged with phenylglyoxal monohydrate (260 mg), 1,3-bis[(2,4,6-trimethyl)phenyl]imidazolinium tetrafluoroborate (50 mg) and tetrahydrofuran (5 g) under a nitrogen atmosphere, and the resulting mixture was stirred while maintaining the temperature of the mixture in a water bath at 25° C. Dry ice (1.0 g) was added to the mixture, and 1,8-diazabicyclo[5,4,0]-7-undecene (23 mg) was further added thereto so as to initiate the reaction, and the mixture was stirred for 2 hours at a room temperature. At 30 minutes and 1 hour following the start of reaction, dry ice (1 g) was added to the reaction mixture, respectively. After completion of the reaction, the solvent was distilled off from the reaction mixture to obtain a yellow solid containing benzoylformic acid.

### Determination of Yield

Methanol (5 g) was added to the obtained yellow solid, and a 10% hexane solution of trimethylsilyldiazomethane was further added thereto, to obtain methyl benzoylformate. A methanol solution containing the obtained methyl benzoylformate was analyzed with a gas chromatography internal standard method to determine the yield of methyl benzoylformate from phenylglyoxal. As a result, the yield was 9%. In other words, benzoylformic acid was obtained from phenylglyoxal at a yield of 9% or more. 55% of phenylglyoxal used as the starting material was recovered.

### Example 5

A 100 ml stainless-steel pressure reaction tube equipped with a magnetic rotor was charged with phenylglyoxal monohydrate (260 mg), 1,3-bis[(2,6-diisopropyl)phenyl]imidazolinium chloride (50 mg) and tetrahydrofuran (5 g) under a nitrogen atmosphere, and the resulting mixture was cooled in a dry ice bath at −70° C. After dry ice (2 g) and 1,8-diazabicyclo[5,4,0]-7-undecene (23 mg) were added to the cooled mixture, the pressure reaction tube was sealed. The reaction was carried out by stirring the resulting mixture for 2 hours at 25° C. After completion of the reaction, the solvent was distilled off from the reaction mixture to obtain a yellow solid containing benzoylformic acid.

### Determination of Yield

Methanol (5 g) was added to the obtained yellow solid, and a 10% hexane solution of trimethylsilyldiazomethane was further added thereto, to obtain methyl benzoylformate. A methanol solution containing the obtained methyl benzoylformate was analyzed with a gas chromatography internal standard method to determine the yield of methyl benzoylformate from phenylglyoxal. As a result, the yield was 7%. In other words, benzoylformic acid was obtained from phenylglyoxal at a yield of 7% or more. 45% of phenylglyoxal used as the starting material was recovered.

### Example 6

A 100 ml stainless-steel pressure reaction tube equipped with a magnetic rotor was charged with phenylglyoxal monohydrate (200 mg), 1,4-dimethyl-1H-1,2,4-triazol-4-ium iodide (25 mg) and tetrahydrofuran (3 g) under a nitrogen atmosphere, and the resulting mixture was cooled in a dry ice bath at −70° C. After dry ice (2 g) and triethylamine (10 mg) were added to the cooled mixture, the pressure reaction tube was sealed. The reaction was carried out by stirring the resulting mixture for 6 hours at 60° C. After completion of the reaction, the solvent was distilled off from the reaction mixture to obtain a yellow solid containing benzoylformic acid.

### Determination of Yield

Methanol (5 g) was added to the obtained yellow solid, and a 10% hexane solution of trimethylsilyldiazomethane was further added thereto, to obtain methyl benzoylformate. A methanol solution containing the obtained methyl benzoylformate was analyzed with a gas chromatography internal standard method to determine the yield of methyl benzoylformate from phenylglyoxal. As a result, the yield was 25%. In other words, benzoylformic acid was obtained from phenylglyoxal at a yield of 25% or more.

### Example 7

A 100 ml stainless-steel pressure reaction tube equipped with a magnetic rotor was charged with phenylglyoxal monohydrate (200 mg), 6,7-dihydro-2-pentafluorophenyl-5H-pyrrolo[2,1,c]-1,2,4-triazolium tetrafluoroborate (27 mg) and tetrahydrofuran (3 g) under a nitrogen atmosphere, and the resulting mixture was cooled in a dry ice bath at −70° C. After dry ice (2 g) and 1,8-diazabicyclo[5,4,0]-7-undecene (10 mg) were added to the cooled mixture, the pressure reaction tube was sealed. The reaction was carried out by stirring the resulting mixture for 6 hours at 40° C. After completion of the reaction, the solvent was distilled off from the reaction mixture to obtain a yellow solid containing benzoylformic acid.

### Determination of Yield

Methanol (5 g) was added to the obtained yellow solid, and a 10% hexane solution of trimethylsilyldiazomethane was further added thereto, to obtain methyl benzoylformate. A methanol solution containing the obtained methyl benzoylformate was analyzed with a gas chromatography internal standard method to determine the yield of methyl benzoylformate from phenylglyoxal. As a result, the yield was 10%. In other words, benzoylformic acid was obtained from phenylglyoxal at a yield of 10% or more.

### Example 8

A 100 ml stainless-steel pressure reaction tube equipped with a magnetic rotor was charged with phenylglyoxal monohydrate (200 mg), 3-(2,6-diisopropyl)phenyl-4,5-dimethylthiazolium chloride (21 mg) and tetrahydrofuran (3 g) under a nitrogen atmosphere, and the resulting mixture was cooled in a dry ice bath at −70° C. After dry ice (2 g) and 1,8-diazabicyclo[5,4,0]-7-undecene (11 mg) were added to the cooled mixture, the pressure reaction tube was sealed. The reaction was carried out by stirring the resulting mixture for 6 hours at 60° C. After completion of the reaction, the solvent was distilled off from the reaction mixture to obtain a yellow solid containing benzoylformic acid.

### Determination of Yield

Methanol (5 g) was added to the obtained yellow solid, and a 10% hexane solution of trimethylsilyldiazomethane was further added thereto, to obtain methyl benzoylformate. A methanol solution containing the obtained methyl benzoylformate was analyzed with a gas chromatography internal standard method to determine the yield of methyl benzoylformate from phenylglyoxal. As a result, the yield was 9%. In other words, benzoylformic acid was obtained from phenylglyoxal at a yield of 9% or more.

### Example 9

A 100 ml stainless-steel pressure reaction tube equipped with a magnetic rotor was charged with methylglyoxal (150 mg), 1,3-bis[(2,6-diisopropyl)phenyl]imidazolium chloride (50 mg) and tetrahydrofuran (3 g) under a nitrogen atmosphere, and the resulting mixture was cooled in a dry ice bath at −70° C. After dry ice (2 g) and 1,8-diazabicyclo[5,4,0]-7-undecene (11 mg) were added to the cooled mixture, the pressure reaction tube was sealed. The reaction was carried out by stirring the resulting mixture for 2 hours at 25° C. After completion of the reaction, the solvent was distilled off from the reaction mixture to obtain a yellow solid containing pyruvic acid.

### Determination of Yield

Methanol (5 g) was added to the obtained yellow solid, and a 10% hexane solution of trimethylsilyldiazomethane was further added thereto, to obtain methyl pyruvate. A methanol solution containing the obtained methyl pyruvate was analyzed with a gas chromatography internal standard method to determine the yield of methyl pyruvate from methylglyoxal. As a result, the yield was 2%. In other words, pyruvic acid was obtained from methylglyoxal at a yield of 2% or more.

### INDUSTRIAL APPLICABILITY

The present invention is useful as a process for producing the α-ketocarboxylic acids which are known as useful compounds for an intermediate in the preparation of pharmaceuticals and agrichemicals since the α-ketocarboxylic acids can be converted to α-amino acids by reductive amination.

1. A process for producing an α-ketocarboxylic acid, comprising a step of oxidizing an α-ketoaldehyde by mixing a base, carbon dioxide, the α-ketoaldehyde and a compound represented by formula (2-1): 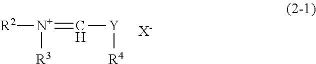
wherein
R² is an alkyl group optionally having a substituent or an aryl group optionally having a substituent;
R³ and R⁴ are independently an alkyl group optionally having a substituent or an aryl group optionally having a substituent, or R³ and R⁴ combine together to form a divalent hydrocarbon group optionally having a substituent or a group of —CH═N— optionally having a substituent;
Y is a group of —S— or —N(R⁵)—, in which R⁵ is an alkyl group optionally having a substituent or an aryl group optionally having a substituent, or R⁵ combines together with R⁴ to form a divalent hydrocarbon group optionally having a substituent; and
X⁻ is an anion.

2. The process according to claim 1, wherein the α-ketoaldehyde is a compound represented by formula (1): 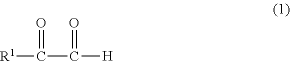
wherein
R¹ is a hydrocarbon group optionally having a substituent or a heteroaryl group optionally having a substituent, and wherein the α-ketocarboxylic acid is a compound represented by formula (3): 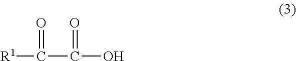
wherein R¹ means the same as defined above.

3. The process according to claim 1, wherein the compound represented by the formula (2-1) is a compound represented by formula (2-2): 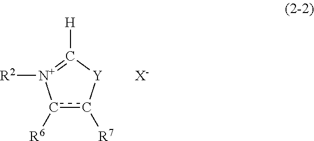
wherein
R² and Y mean the same as defined above;
R⁶ and R⁷ are independently a hydrogen atom, an alkyl group optionally having a substituent or an aryl group optionally having a substituent, or R⁶ and R⁷ are bonded to each other to form a ring together with carbon atoms to which they attach, or R⁷ combines together with R⁵ to form a divalent hydrocarbon group optionally having a substituent; 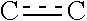
represents a carbon-carbon single bond or a carbon-carbon double bond; and
X⁻ means the same as defined above, or a compound represented by formula (2-3): 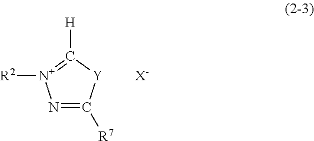
wherein
R² and Y mean the same as defined above;
R⁷ is a hydrogen atom, an alkyl group optionally having a substituent or an aryl group optionally having a substituent, or R⁷ combines together with R⁵ to form a divalent hydrocarbon group optionally having a substituent; and
X⁻ means the same as defined above.

4. The process according to claim 3, wherein the compound represented by the formula (2-1) is a compound represented by the formula (2-2).

5. The process according to claim 4, wherein in the formula (2-2) Y is a group of —N(R⁵)—, R² and R⁵ are independently a 2,6-disubstituted phenyl group, R⁶ and R⁷ are both a hydrogen atom, and 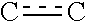
represents a carbon-carbon double bond.

6. The process according to claim 1, wherein the base is at least one base selected from the group consisting of organic bases, alkali metal carbonates and alkaline earth metal carbonates.

7. A process for producing an α-ketocarboxylic acid, comprising a step of oxidizing an α-ketoaldehyde in the presence of carbon dioxide and a compound obtained by bringing a base into contact with a compound represented by formula (2-1): 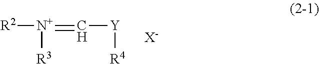
wherein
R² is an alkyl group optionally having a substituent or an aryl group optionally having a substituent;
R³ and R⁴ are independently an alkyl group optionally having a substituent or an aryl group optionally having a substituent, or R³ and R⁴ combine together to form a divalent hydrocarbon group optionally having a substituent or a group of —CH═N— optionally having a substituent;
Y is a group of —S— or —N(R⁵)—, in which R⁵ is an alkyl group optionally having a substituent or an aryl group optionally having a substituent, or R⁵ combines together with R⁴ to form a divalent hydrocarbon group optionally having a substituent; and
X⁻ is an anion.

8. The process according to claim 7, wherein the α-ketoaldehyde is a compound represented by formula (1): 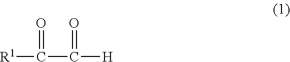
wherein
R¹ is a hydrocarbon group optionally having a substituent or a heteroaryl group optionally having a substituent,
and wherein the α-ketocarboxylic acid is a compound represented by formula (3): 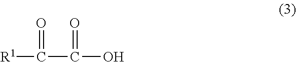
wherein R¹ means the same as defined above.

9. The process according to claim 7, wherein the compound represented by the formula (2-1) is a compound represented by formula (2-2): 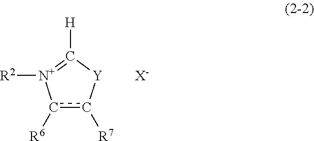
wherein
R² and Y mean the same as defined above;
R⁶ and R⁷ are independently a hydrogen atom, an alkyl group optionally having a substituent or an aryl group optionally having a substituent, or R⁶ and R⁷ are bonded to each other to form a ring together with carbon atoms to which they attach, or R⁷ combines together with R⁵ to form a divalent hydrocarbon group optionally having a substituent; 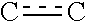
represents a carbon-carbon single bond or a carbon-carbon double bond; and
X⁻ means the same as defined above;
or a compound represented by formula (2-3): 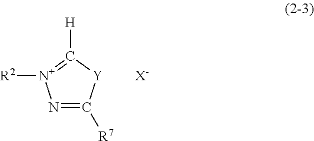
wherein
R² and Y mean the same as defined above; and
R⁷ is a hydrogen atom, an alkyl group optionally having a substituent or an aryl group optionally having a substituent, or R⁷ combines together with R⁵ to form a divalent hydrocarbon group optionally having a substituent; and
X⁻ means the same as defined above.

10. The process according to claim 9, wherein the compound represented by the formula (2-1) is a compound represented by the formula (2-2).

11. The process according to claim 10, wherein in the formula (2-2) Y is a group of —N(R⁵)—, R² and R⁵ independently a 2,6-disubstituted phenyl group, R⁶ and R⁷ are both a hydrogen atom, and 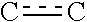
represents a carbon-carbon double bond.

12. The process according to claim 7, wherein the base is at least one base selected from the group consisting of organic bases, alkali metal carbonates and alkaline earth metal carbonates.
